(19) United States
(12) Patent Application Publication
(10) Pub. No.: US 2023/0086111 A1
(43) Pub. Date: Mar. 23, 2023

(54) A MULTI FOCAL ENDOSCOPE

(71) Applicant: 270 SURGICAL LTD., Netanya (IL)

(72) Inventors: Avraham LEVY, Kfar Shmaryahu (IL); Victor LEVIN, Haifa (IL)

(21) Appl. No.: 17/798,416

(22) PCT Filed: Mar. 1, 2021
(86) PCT No.: PCT/IL2021/050224

§ 371 Date: Aug. 9, 2022

Related U.S. Application Data
(60) Provisional application No. 62/985,980, filed on Mar. 6, 2020.

(51) Int. Cl. A61B 1/00

(52) U.S. Cl. CPC A61B 1/00096; A61B 1/00009; A61B 1/00045; A61B 1/00194

(57) ABSTRACT

Provided is an endoscope having a tip at a distal section thereof, the tip includes a plurality of imaging units, at least one of the imaging units includes at least two optical lens assemblies and at least one optical sensor associated with the at least two optical lens assemblies, wherein each optical lens assembly has a different depth of field, thereby allowing to obtain a multi-focal image of a body cavity. Further provided are systems comprising the endoscope and methods of using the same in various endoscopic procedures.

### TECHNICAL FIELD

The present disclosure relates generally to a multiple focal endoscope having a plurality of imaging units, configured to provide multi focal image(s) at varying depth of field relative to a distal tip of the endoscope.

### BACKGROUND

An endoscope is a medical device used to image an anatomical site (e.g. a anatomical/body cavity, a hollow organ). Unlike some other medical imaging devices, the endoscope is inserted into the anatomical site (e.g. through small incisions made on the skin of the patient). An endoscope can be employed not only to inspect an anatomical site and organs therein (and diagnose a medical condition in the anatomical site) but also as a visual aid in surgical procedures. Medical procedures involving endoscopy include laparoscopy, arthroscopy, cystoscopy, ureterostomy, and hysterectomy.

When performing such medical procedures it would be advantageous to obtain a an enhanced view of the anatomical site, at different distances, to more easily and reliably identify, magnify and visualize objects of interest during the endoscopic procedures, to thereby increase safety and efficiency.

There is thus a need in the art for an endoscope system having more than one imaging units, wherein at least one of the imaging unit is multi-focal, capable of providing an enhanced field of view which includes enhanced depth of view of objects viewed during the endoscopic procedure.

### SUMMARY

Aspects of the disclosure, according to some embodiments thereof, relate to endoscope having a plurality of imaging units at the endoscope distal tip, wherein at least one of the imaging units includes more than one optical lens assemblies, wherein each optical lens assembly includes a different depth of view, consequently allowing to provide multi-focal views of area/region(s) of interest of a subject’s body.

According to some embodiments, there is provided an advantageous multi-focal, multi-imaging units endoscope systems that may be used to more precisely visualize (optionally at 3D view) and identify objects of interest at a large and varying depth of field during endoscopic procedures, to thereby result in more accurate and safe medical procedures.

According to some embodiments, the devices and systems disclosed herein are advantageous, as they allow obtaining, visualizing, identifying and/or magnifying objects or areas/regions of interest in a cost effective and efficient manner, during the medical procedure, by utilizing imaging units having multiple lenses/lens assemblies, while being small and compact enough to fit within the limited space of the endoscope tip, and without compromising image quality.

According to some embodiments, the devices and systems disclosed herein are further advantageous, as they allow obtaining images at a wide range of depth of field and/or wide varying range of working distances, allowing a user to perceive an enhanced view of the region of interest.

According to some embodiments, there is provided an endoscope tip having at least two imaging units, wherein at least one of said imaging units includes more than one lens assembly, each of the lens assembly is configured to provide an image at a different focal distance, to thereby allow forming a multi-focal view of a wide range of working distances and/or depths of fields.

According to some embodiments, there is provided an endoscope tip having at least two imaging units, wherein at least one of imaging unit is configured to provide images from different/varying working distances and/or depth of fields distances (i.e., it is multi focal).

According to some embodiments, there is provided an endoscope tip having at least two, at least three imaging units (optical assemblies), wherein at least one of said imaging unit include more than one lens assembly, each of the lens assembly is configured to provide an image having a different focal distance, to thereby form a multi-focal or 3D view of an area of interest.

According to some embodiments, there is provided an endoscope distal tip having a plurality of imaging units, at least one of said imaging units comprises at least two optical lens assemblies and at least one optical sensor associated with the at least two optical lens assemblies, wherein each optical lens assembly comprises a different depth of field, thereby allowing to obtain a focused image of at least one body/anatomical cavity region close to the endoscope tip and at least one cavity region farther away from the endoscope tip within the body cavity.

According to some embodiments, there is provided an endoscope distal tip having a plurality of imaging units, at least one of said imaging units comprises at least two optical lens assemblies and at least one optical sensor associated with the at least two optical lens assemblies, wherein each optical lens assembly comprises a different depth of field, thereby allowing to obtain a focused image of a body region in a body cavity, at a varying working distance and/or depth of field relative to the endoscope distal tip.

According to some embodiments, there is provided a distal tip of an endoscope having at least two imaging units, at least one of said imaging units includes at least two optical lens assemblies and at least one optical sensor associated with the at least two optical lenses, wherein each optical lens assembly comprises a different depth of field, to thereby form a multi-focal or 3D view of an area/region of interest in which the endoscope distal tip resides.

According to some embodiments, at least one of the imaging units may include three optical lens assemblies, each of the lens assemblies is associated with an optical sensor, wherein each of said optical lens assemblies has a different depth of field, to thereby from a multi-focal or 3D view.

In some embodiments the optical lens assemblies of an imaging unit have a common field of view (FOV). In some embodiments, the optical lens assemblies of an imaging unit are associated with on optical sensor. In some embodiments, the three optical lens assemblies are different with respect of size, shape and/or composition.

According to some embodiments, there is provided an endoscope which includes a handle, and a compatible shaft having a tip as disclosed herein, at a distal section of the shaft.

According to some embodiments, the endoscope may include a plurality of imaging units positioned at the tip at a distal section of the shaft, wherein at least one of the imaging units includes more than one lens or lens assemblies. In some embodiments, the imaging units may provide combined and consistent panoramic view, at varying working distances and/or depth of fields.

According to some embodiments, the endoscope may include at least one imaging unit having at least two optical lenses/lens assemblies, and at least one illumination component located at the shaft distal section.

According to some embodiments, the endoscope may include at least two imaging units, wherein at least one of the imaging units include more than one optical lens/lens assembly.

In some embodiments, the endoscope distal tip may include at least two imaging units, wherein at least one of the imaging units may include more than one lens/lens assembly, each having a different depth of field. According to some embodiments, the at least two imaging units of the endoscope may include a front imaging unit (that may include one or more optical lens assemblies, as disclosed herein) on a distal tip of the shaft and a first side-imaging unit (that may include one or more optical lens assemblies, as disclosed herein). According to some embodiments, the at least two imaging units may include a second side-imaging (that may include one or more optical lens assemblies, as disclosed herein), wherein the first side-imaging unit and the second side-imaging unit are positioned on opposite sides of the distal tip of the endoscope, and wherein the second side-imaging unit is positioned distally relative to the first side imaging unit.

According to some embodiments, the at least two imaging units may provide at least about 270 degrees horizontal field of view (FOV) of a region of interest within an anatomical cavity into which the elongated shaft is inserted, wherein at least one of the imaging units has more than one optical lens assembly, each having a different depth of field, to thereby provide a multi-focal or 3D image of the anatomical cavity.

According to some embodiments, the endoscope may further include at least one illumination component that may be a discrete light source, such as, for example, a light emitting diode (LED). According to some embodiments, the at least one illumination component is or comprises a discrete light source.

According to some embodiments, there is provided an endoscope tip which includes a plurality of imaging units, at least one of said imaging units comprises at least two optical lens assemblies and at least one optical sensor associated with the at least two optical lens assemblies, wherein each optical lens assembly comprises a different depth of field, thereby allowing to obtain a focused image of a body part at a varying depth of field relative to the tip.

According to some embodiments, at least one optical sensor may be associated with an image processor. According to some embodiments, the sensor may be selected from CMOS and CCD.

According to some embodiments, the distal tip may further include one or more illumination components associated with the at least one imaging unit.

According to some embodiments, each optical assembly may be associated with a respective optical sensor.

According to some embodiments, at least one of the imaging units may include at least three optical lens assemblies.

According to some embodiments, the endoscope tip may include at least two imaging units.

According to some embodiments, the plurality of imaging units may include a front facing imaging unit and a first side facing imaging unit.

According to some embodiments, the at least two imaging units may further include a second side-imaging unit, wherein the first imaging unit and the second imaging unit may be positioned on opposite sides.

According to some embodiments, the at least two imaging units may provide at least about 270 degrees horizontal field of view (FOV) of a region of interest within an anatomical body cavity into which the distal tip is inserted, wherein at least one of the field of views comprises a multi focal image.

According to some embodiments, at least two of the imaging units may each include at least three optical lens assemblies.

According to some embodiments, the optical lens assemblies of an imaging unit may be arranged in the form of a triangle, wherein the distance between the center of the optical lenses is smaller than about 3 millimeters.

According to some embodiments, the minimal distance between the optical lens assemblies of the imaging unit may be smaller than a minimal distance between an optical lens assembly with a shortest depth of field and the region of interest.

According to some embodiments, the optical lens assemblies of an imaging unit may be arranged in horizontal or vertical line relative to each other.

According to some embodiments, a first optical lens assembly may be configured to provide a focused image in a depth of field of about 2-10 millimeters.

According to some embodiments, the minimal distance between the optical lens assemblies of the imaging unit may be smaller than the minimal distance between the first (smallest) optical lens assembly having the shortest depth of field and the region of interest.

According to some embodiments, a second optical lens assembly may be configured to provide a focused image in a depth of field of about 5-20 millimeters.

According to some embodiments, a third optical lens assembly may be configured to provide a focused image in a depth of field of about 15-500 millimeters.

According to some embodiments, the varying depth of field relative to the tip is in the range of about 2-500 millimeters.

According to some embodiments, the at least one illumination component may be or may include a discrete light source.

According to some embodiments, each of the optical lens assemblies may include a discrete illumination component.

According to some embodiments, at least one of the optical lens assemblies may include autofocus capabilities.

According to some embodiments, there is provided an endoscope which includes the tip as disclosed herein, at a distal section of an elongated shaft of the endoscope.

According to some embodiments, shaft is configured to be inserted to a region of interest within an anatomical body cavity.

According to some embodiments, the shaft may be rigid, semi-rigid or flexible.

According to some embodiments, the endoscope disclosed herein may be used in endoscopic procedures selected from: laparoscopy, colonoscopy, genecology arthroscopy, cystoscopy, ureterostomy, hysterectomy, renal procedures, urological procedures, nasal procedure and orthopedic procedures. Each possibility is a separate embodiment.

According to some embodiments, there is provided a medical imaging system which includes the endoscope disclosed herein, and a display configured to display the images and/or video generated by the one or more of the imaging units.

According to some embodiments, the system may further include a processing unit configured to receive images obtained from the optical lens assemblies of the imaging units and generate in real time a focused image at a varying depth of field.

According to some embodiments, the generated focused image is a 3D image of a body cavity, in which the endoscope tip resides.

According to some embodiments, there is provided a method for obtaining a focused image of a region of interest at a varying depth of fields relative to the tip, the method may include the steps of:
inserting into the region of interest an endoscope shaft having a tip at a distal section thereof, the tip includes a plurality of imaging units, at least one of said imaging units comprises at least two optical lens assemblies and at least one optical sensor associated with the at least two optical lens assemblies, wherein each optical lens assembly has a different depth of field; and
generating a focused image of the region of interest at a varying depth of field relative to the tip.

According to some embodiments, the varying depth of field is in the range of about 2-500 millimeters.

According to some embodiments, the generated focused image is generated in real time by a processing unit configured to generate the focused image based on the images obtained from the at least two optical lens assemblies of the imaging units.

According to some embodiments, the focused image is a multi-focal image. According to some embodiments, the focused image is a 3D image.

According to some embodiments, the method may further include displaying the focused image on a display.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1 - shows a schematic, perspective view of an endoscope including a handle, and an elongated shaft having a distal tip, according to some embodiments;

FIG. 2 - schematically depicts a medical imaging system including an endoscope having a plurality of imaging units, according to some embodiments;

FIG. 3 - schematically depicts an elongated shaft of an endoscope, and a field-of-view provided by imaging units positioned in a distal section (tip) of the elongated shaft, according to some embodiments;

FIG. 4A - shows a perspective view of a front and side imaging units disposed at a distal tip of an endoscope, according to FIG. 3;

FIG. 4B - shows a schematic zoom-in view of an imaging unit, according to FIG. 4A;

FIG. 4C - shows a schematic zoom-in view of an imaging unit, according to FIG. 4A;

FIG. 4D - shows a schematic zoom-in view of an imaging unit, according to FIG. 4A; and

FIG. 5 - shows a method for obtaining a focused image of a body cavity/area of interest at varying depth of field, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

According to some embodiments, there is provided herein an advantageous endoscope having two or more imaging units at a distal end (tip) thereof, wherein at least one of said imaging units have two or more optical lens assemblies, associated with at least one image sensor, wherein each of the optical lens assemblies are configured to provide images at a different depth of field, to thereby obtain multi focal or 3D images of body regions in which the distal tip of the endoscope resides.

As used herein, the term “imaging unit” refers to a unit which includes one or more optical lens assemblies, associated with at least one optical sensor, wherein each of the optical lens assemblies in an imaging unit is configured to have a different depth of field, a different focal length, an equal or a different field of view and/or an equal or different direction of view. In some embodiments, each of the optical lens assemblies is associated with an optical sensor. In some embodiments, an imaging unit includes one or more cameras (wherein each camera has optical lens assemblies associated with an optical/imaging sensor).

As used herein, the terms “lens”, “optical lens assembly” and “lens assembly” refer to an optical lens associated with a suitable image sensor, capable of forming an image. In some embodiments, two or more lenses may share a common optical/image sensor. In some embodiments, each of the lens assemblies in an imaging unit may be different with respect of size, composition, shape, focal length, depth of field, visual field, and the like.

As used herein the terms “optical sensor” “imaging sensor” and “image sensor” may interchangeably be used. The terms refer to a sensor as known in the art which conveys information from the optical lens assembly to make/generate an image. In some embodiments, the image sensor may be of the type of charge coupled device (CCD) or a complementary metal oxide semiconductor (CMOS).

As used herein the terms “depth of field” and “depth of view”, may interchangeably be used. The terms refer to a range of distances through which an object/region being imaged may move in or out of the plane of best focus while maintaining an acceptable level of contrast at a particular spatial frequency or resolution.

As used herein, the term “working distance” relates to a specific distance through which an object/region being imaged is in the plane of best focus. In some embodiments, a working distance is a distance between the end of a lens and the object/region being imaged. In some embodiments, a working distance is a value within a range of values of a corresponding depth of field.

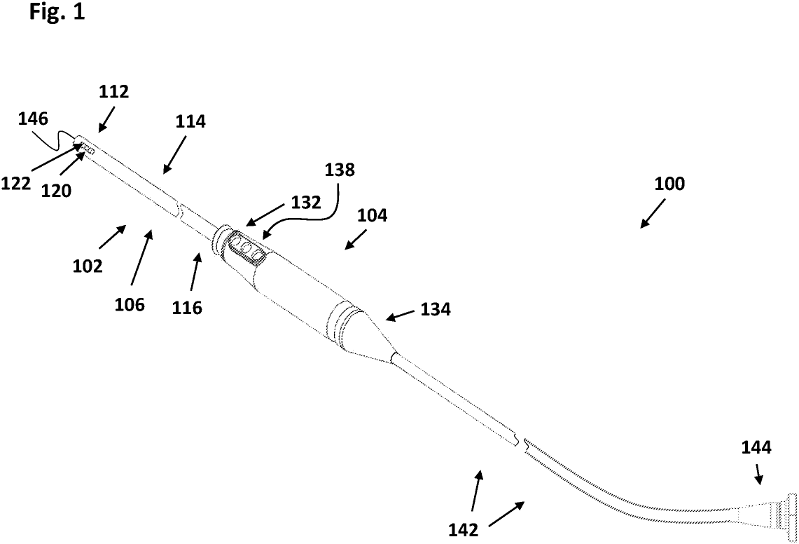

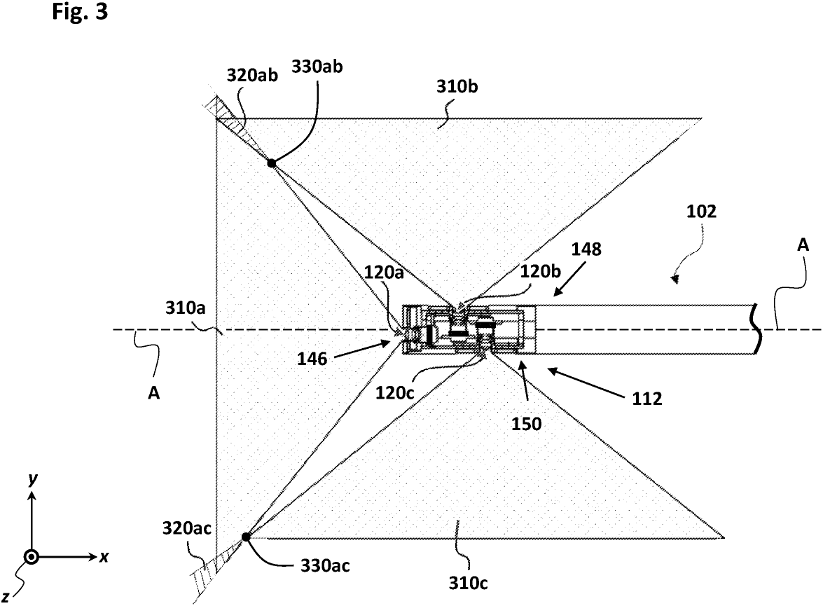

Reference is now made to FIG. 1, which schematically depict a rigid endoscope, according to some embodiments. As shown in FIG. 1, endoscope 100 includes an elongated shaft 102, configured to be inserted into an anatomical site (e.g. an anatomical cavity), and a handle 104, configured to be held by a user (e.g. a surgeon) of endoscope 100 and to facilitate guiding and manipulation of elongated shaft 102 (particularly a distal section thereof) within the anatomical site. Shaft 102 includes a shaft body 106, e.g. a rigid tubular member. Shaft 102 includes a shaft distal section 112, a shaft central section 114, and a shaft proximal section 116 (i.e. a distal section, a central section, and a proximal section, respectively, of shaft 102). Shaft distal section 112 includes at least two imaging units 120 (e.g. a front imaging unit, as seen for example in FIG. 3, and at least one side imaging unit) and illumination components 122, such as light emitting diodes (LEDs). At least one of imaging units 120 may include two or more optical lens assemblies, associated with at least one image sensor, as further detailed below. According to some embodiments, each of illumination components 122 is or includes a discrete light source. According to some embodiments, wherein illumination components 122 include LEDs, the LEDs may include, for example, one or more white light LEDs, infrared LEDs, near infrared LEDs, an ultraviolet LED, and/or a combination thereof. It is noted that in embodiments wherein illumination components include LEDs configured to produce light outside the visible spectrum (e.g. an infrared spectrum, a UV spectrum), imaging units 120 may include suitable sensors configured to detect such type of light (e.g. infrared light, ultraviolet). That is, imaging units 120 will have capacities of e.g. infrared cameras and so on. According to some embodiments, the illumination components may include the distal tips of respective optical fibers (not shown).

The handle 104 may include a user control interface 138 configured to allow a user to control endoscope 100 functions. User control interface 138 may be functionally associated with imaging units 120 and illumination components 122 via an electronic coupling between shaft 102 and handle 104. According to some embodiments, user control interface 138 may allow, for example, to control zoom, focus, multifocal views, record/stop recording, freeze frame functions, etc., of imaging units 120 and/or to adjust the light intensity provided by illumination components 122.

According to some embodiments, at least one of imaging units 120 may include at least two lens assemblies (each having a different focal length configured to provide image of a different depth of field) and at least one sensor, such as a charge coupled device (CCD) image sensor or a complementary metal oxide semiconductor (CMOS) image sensor. Imaging units 120 may be configured to provide a continuous/panoramic/surround multifocal (3D) field-of-view (FOV), as elaborated on below in the description of FIG. 3, in a multifocal.

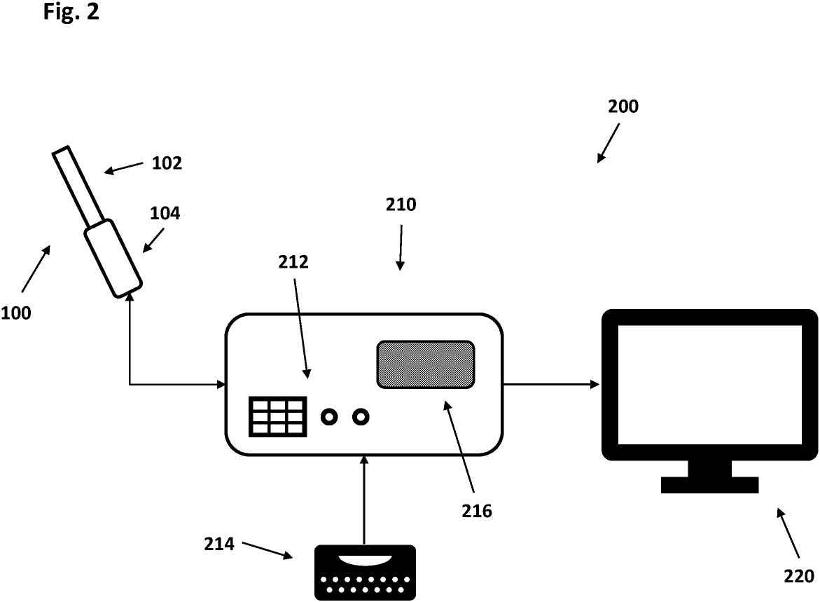

Reference is now made to FIG. 2, which schematically depicts a medical imaging system 200, according to some embodiments. Medical imaging system 200 includes endoscope 100, a main control unit 210, and a monitor 220. According to the convention adopted herein, a same reference numeral in FIGS. 1, 2 and 3, refers to the same object (e.g. device, element).

As shown in FIG. 2, Endoscope 100 and monitor 220 may each be functionally associated with main control unit 210. Main control unit 210 includes processing circuitry (e.g. one or more processors and memory components) configured to process (digital data) from imaging units 120 (not shown in FIG. 2 but depicted in FIG. 1), such as to display the captured image, in particular in multi-focal or 3D, and video streams on monitor 220. In particular, the processing circuitry may be configured to process the digital data received from each of imaging units 120, such as to produce therefrom video files/streams providing a 3D, panoramic/surround view of the anatomical site, as explained below in the description of FIG. 3. According to some embodiments, the processing circuitry may be configured to process the data received from imaging units 120 to produce a combined video stream providing a continuous and consistent (seamless) panoramic view of the anatomical site.

Main control unit 210 may include a user interface 212 (e.g. buttons and/or knobs, a touch panel, a touch screen) configured to allow a user to operate main control unit 210 and/or may allow control thereof using one or more input devices 214, e.g. an external user control interface connectable thereto such as a keyboard, a mouse, a portable computer, and/or even a mobile computational device e.g. a smartphone or a tablet. According to some embodiments, input devices 214 may include a voice controller. According to some embodiments, main control unit 210 may further be configured to partially or even fully operate imaging units 120 and illumination components 122 (shown in FIG. 1). Some operational aspects may be operated automatically, for example, according to some embodiments, the supply of power to endoscope 100 components, such as imaging units 120 and illumination components 122, while other operational aspects or functions may be operated using user interface 212 and/or input devices 214. According to some embodiments, main control unit 210 may include a display 216 (for example, the touch screen and/or another screen) for presenting information regarding the operation of endoscope 100, such as the brightness levels of imaging units 120, zoom options, focus, and the like. According to some embodiments, wherein display 216 is a touch screen, display 216 may further allow controlling for example, the zoom, focus, multifocal imaging, selecting images from specific lens assemblies, compiling images from various lens assemblies, creating a multifocal image, record/stop recording functions, freeze frame function, and/or the brightness of imaging units 120, and/or to adjust the light intensity of illumination components 122. According to some embodiments, the choice of information presented may be controlled using user interface 212, user control interface 138, and/or input devices 214.

According to some embodiments, endoscope 100 is functionally associated with main control unit 210 via a utility cable 142 (shown in FIG. 1), connected to or configured to be connected to handle proximal section 134, and further configured to be connected to main control unit 210 (via, for example, a plug 144 or a port). Utility cable 142 may include at least one data cable for receiving video signals from imaging units 120, and at least one power cable for providing electrical power to imaging units 120 and to illumination components 122, as well as to operationally control parameters of imaging units 120 and illumination components 122, such as the light intensity. Additionally or alternatively, according to some embodiments, endoscope 100 may include a wireless communication unit (e.g. a Bluetooth antenna or Wi-Fi) configured to communicatively associate endoscope 100 with main control unit 210. According to some embodiments, endoscope 100 is configured to be powered by a replaceable and/or rechargeable battery included therein, i.e. inside handle 104. According to some embodiments, wherein illumination components 122 include the distal tips of optical fibers and wherein the light source(s) is positioned in main control unit 210, cable 142 will also include one or more optical fibers configured to guide the light produced by the light source(s) to an optical fiber(s) in handle 104, wherefrom the light will be guided to optical fibers in shaft 102.

Monitor 220 is configured to display images and, in particular, to display multifocal stream videos captured by imaging units 120, and may be connected to main control unit 210 by a cable (e.g. a video cable) or wirelessly. According to some embodiments, monitor 220 may be configured to display thereon information regarding the operation of endoscope 100, as specified above. According to some embodiments, monitor 220, or a part thereof, may function as a touch screen. According to some such embodiments, the touch screen may be used to operate main control unit 210. According to some embodiments, images/videos from different imaging units (from imaging units 120) or from different lens assemblies of the different imaging units, may be displayed separately (e.g. side-by-side, picture on picture, in an equal aspect ratio, in an un-equal aspect ratios, in multiple copies of one or more of the video streams, and the like) on monitor 220, and/or may be presented as a single panoramic/surround, optionally multifocal or 3D image/video. According to some embodiments, user interface 212 and/or input devices 214 and/or user control interface 138 are configured to allow switching between images/videos corresponding to different FOVs (of different imaging units) and/or of different field of views (obtained from different lens assemblies of one or more imaging units). For example, according to some embodiments, wherein imaging units 120 include a front imaging unit 120a, a first side imaging unit 120b, and a second side imaging units 120c: switching between footage(s) captured by one or more lens assemblies from front imaging unit 120a to footage(s) captured by one or more lens assemblies of first side imaging unit 120b, switching between footage(s) captured by one or more lens assemblies of front imaging unit 120a to footage(s) captured by one or more lens assemblies of second side imaging units 120c, or switching between panoramic/surround video(s) generated from the footage(s) of all of imaging units 120a, 120b, and 120c to footage captured by one of imaging units 120a, 120b, or 120c. Imaging units 120a, 120b, and 120c are depicted together in FIG. 3. Optical lens assemblies of one or more of the imaging units are not shown in FIG. 3. According to some embodiments, main control unit 210 may be associated with a plurality of monitors, such as monitor 220, thereby allowing displaying different videos and images on each. For example, main control unit 210 may be associated with four monitors, such as to allow displaying videos from each of imaging units 120a, 120b, 120c on three of the monitors, respectively, and a panoramic video (corresponding to the combination of the three videos) on the fourth monitor, which may be wider than the other three.

The field-of-view (FOV) provided by endoscope 100 is the combination of the respective FOVs provided by each of imaging units 120. Imaging units 120 may be configured to provide a continuous and consistent FOV, or at least a continuous and consistent horizontal FOV (HFOV), wherein each of the views may be multifocal view (providing images at varying depth of fields), depending on the number and composition of the optical lens assemblies of each of the respective imaging units.

Reference is now made to FIG. 3, which schematically depicts shaft distal section 112 (of shaft 102) and a combined HFOV provided by a front imaging unit 120a, a first side imaging unit 120b, and a second side imaging unit 120c, according to some embodiments. Front imaging unit 120a is positioned within shaft distal section 112 on a front surface 146 of the distal tip (not numbered) of shaft distal section 112, with one or more lens assemblies (having one or more optical sensors) of front imaging unit 120a being exposed on front surface 146. First side imaging unit 120b is positioned within shaft distal section 112 on a first side surface 148 of the distal tip (not numbered), with one or more lens assemblies (not numbered) of first side imaging unit 120b being exposed on first side surface 148. Second side imaging unit 120c is positioned within shaft distal section 112 on a second side surface 150 of the distal tip (not numbered), with one or more lens assemblies (not numbered) of second side imaging unit 120c being exposed on second side-surface 150. First side surface 148 is opposite to second side surface 150. According to some embodiments, first side imaging unit 120b and second side imaging unit 120c are not positioned back-to-back. According to some embodiments, the distances between the center points of the imaging units are adjusted based on the size, type and/or number of the optical lenses of each of the imaging units. According to some embodiments, the distance of first side imaging unit 120b (i.e. the center of a lens assembly of first side- imaging unit 120b) and front surface 146 is between about 5 millimeters to about 20 millimeters and the distance between the center point of first side imaging unit 120b and the center-point of second side imaging unit 120c may be up to about 10 millimeters.

In some embodiments, a combined/panoramic/surround HFOV may be formed by a front HFOV 310a, a first side HFOV 310b, and a second side HFOV 310c of front imaging units 120a, first side imaging unit 120b, and second side imaging unit 120c, respectively. Each of HFOVs 310a, 310b, and 310c lies on the xy-plane. HFOV 310a is positioned between HFOVs 310b and 310c and overlaps with each. A first overlap area 320ab corresponds to an area whereon HFOVs 310a and 310b overlap. In other words, first overlap area 320ab is defined by the intersection of the xy-plane with the overlap region (volume) of the FOVs of front imaging unit 120a and first side imaging unit 120b. Similarly, a second overlap area 320ac corresponds to an area whereon HFOVs 310a and 310c overlap. A first intersection point 330ab is defined as the point in first overlap area 320ab which is closest to front imaging unit 120a. It is noted that first intersection point 330ab also corresponds to the point in first overlap area 320ab which is closest to first side-imaging unit 120b. Similarly, a second intersection point 330ac is defined as the point in second overlap area 320ac which is closest to front imaging unit 120a. It is noted that second intersection point 330ac also corresponds to the point in second overlap area 320ac which is closest to second side imaging unit 120c.

The combined HFOV (of imaging units 120a, 120b, and 120c) is continuous since the panoramic view provided thereby does not contain any gaps (as would have been the case had HFOV 310a not overlapped with at least one of HFOVs 310b and 310c). Further, the combined HFOV is consistent (i.e. seamless) in the sense that the magnifications of the various optical lenses assemblies of each of imaging units 120a, 120b, and 120c are compatible such that the view of objects (e.g. organs or surgical tools), or parts of objects, in the overlap areas are not distorted and the (overall) combined HFOV merges the combined multifocal HFOVs of each front HFOV 310a and first side HFOV 310b, and front HFOV 310a and second side HFOV 310c, in a seamless manner. Thus, magnifications provided by lens assemblies of first side imaging unit 120b may be different than the magnifications provided by the optical lens assemblies of front imaging unit 120a to compensate for first intersection point 330ab being closer to front imaging unit 120a than to first side-imaging unit 120b. According to some embodiments, the combined HFOV spans between about 200 degrees to about 270 degrees, between about 240 degrees to about 300 degrees, or between about 240 degrees to about 340 degrees. Each possibility is a separate embodiment. According to some embodiments, the combined HFOV spans at least about 270 degrees. According to some embodiments, for example, each of HFOVs 310a, 310b, and 310c may measure between about 85 degrees to about 120 degrees, between about 90 degrees to about 110 degrees, or between about 85 degrees to about 110 degrees, between about 95 degrees to about 120 degrees. Each possibility corresponds to separate embodiments. According to some embodiments, additionally, or alternatively, the relative location of at least one of the front lens of imaging unit 120a and at least one of the side lens of imaging unit 120b and/or 120c may also affect the combined FOV. In some embodiments, the distance between front lens of 120a the entrance aperture of a front lens of imaging unit 120a, and the optical axis of side lens of imaging unit 210b and/or 210c may be smaller than about 23 -27 millimeters. In some embodiments, the distance may be smaller than about 20 millimeters. In some embodiments, the distance may be smaller than about 23 millimeters. In some embodiments, the distance may be smaller than about 25 millimeters. In some embodiments, the distance may be smaller than about 27 millimeters. In some embodiments, the distance may be smaller than about 30 millimeters.

According to some embodiments, shaft 102 may measure between about 100 millimeters and about 500 millimeters in length, and shaft body 106 may have a diameter measuring between about 2.5 millimeters and about 15 millimeters. According to some embodiments, front imaging unit 120a may be offset relative to a longitudinal axis A, which centrally extends along the length of shaft 102. According to some embodiments, the distance between second side imaging unit 120c and front surface 146 is greater than the distance between first side imaging unit 120b and front surface 146.

According to some embodiments, front imaging unit 120a may be offset relative to the longitudinal axis A by up to about 0.05 millimeters, up to about 0.1 millimeters, up to about 0.5 millimeters, up to about 1.0 millimeters, up to about 1.5 millimeters, up to about 5.0 millimeters, or up to about 7.0 millimeters. Each possibility corresponds to separate embodiment. According to some embodiments, for example, front imaging unit 120a may be offset relative to the longitudinal axis A by between about 0.05 millimeters to about 0.1 millimeters, about 0.5 millimeters to about 1.5 millimeters, about 1.0 millimeter to about 5.0 millimeters, about 1.5 millimeters to about 5.0 millimeters, or about 1.0 millimeters to about 7.0 millimeters. Each possibility corresponds to separate embodiments. According to some embodiments, first side imaging unit 120b may be positioned at a distance of up to about 1.0 millimeters, up to about 5.0 millimeters, or up to about 15.0 millimeters from front surface 146. Each possibility corresponds to separate embodiments. According to some embodiments, second side imaging unit 120c may be positioned at a distance of up to about 1.0 millimeters, up to about 5.0 millimeters, up to about 15.0 millimeters, or up to about 25.0 millimeters from front surface 146, such as to optionally be positioned farther from front surface 146 than first-side imaging unit 120b. Each possibility corresponds to separate embodiments. According to some embodiments, the positioning of imaging units 120 on shaft distal section 112 is selected such as to minimize the space occupied by imaging units 120 and reduce the diameter of shaft distal section 112, while affording a continuous and consistent HFOV of about 200 degrees, of about 240 degrees, of at least about 270 degrees.

According to some embodiments, each of imaging unit 120 is associated with one or more respective illumination component from illumination components 122, which is configured to illuminate the FOVs of the imaging units. Thus, according to some embodiments, front imaging unit 120a may be associated with a respective one or more front illumination component (not numbered), first side imaging unit 120b may be associated with a respective one or more first side illumination component, and second side imaging unit 120c may be associated with a respective one or more second side illumination component.

According to some embodiments, not depicted in the figures, imaging units 120 include only two imaging units, both of which are side imaging units, wherein at least one of these imaging units includes two or more optical lens assemblies. In such embodiments, shaft distal section 112 may taper in the distal section, such that the imaging unit provide a continuous HFOV. According to some embodiments, not depicted in the figures, imaging unit 120 include only two imaging unit: a front imaging unit and a side imaging unit, wherein at least one of said imaging units includes two or more optical lens assemblies associated with at least one optical sensor.

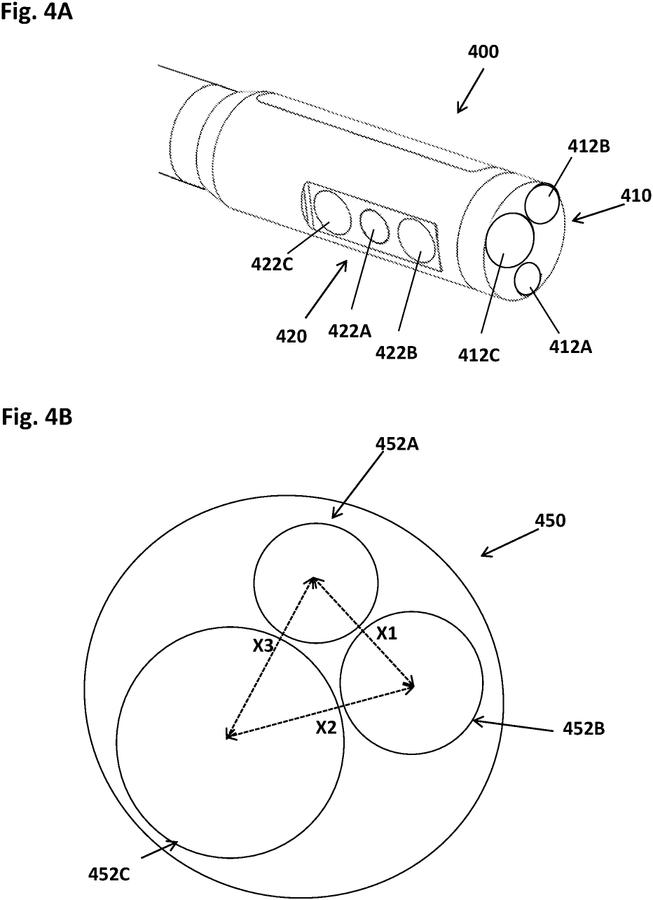

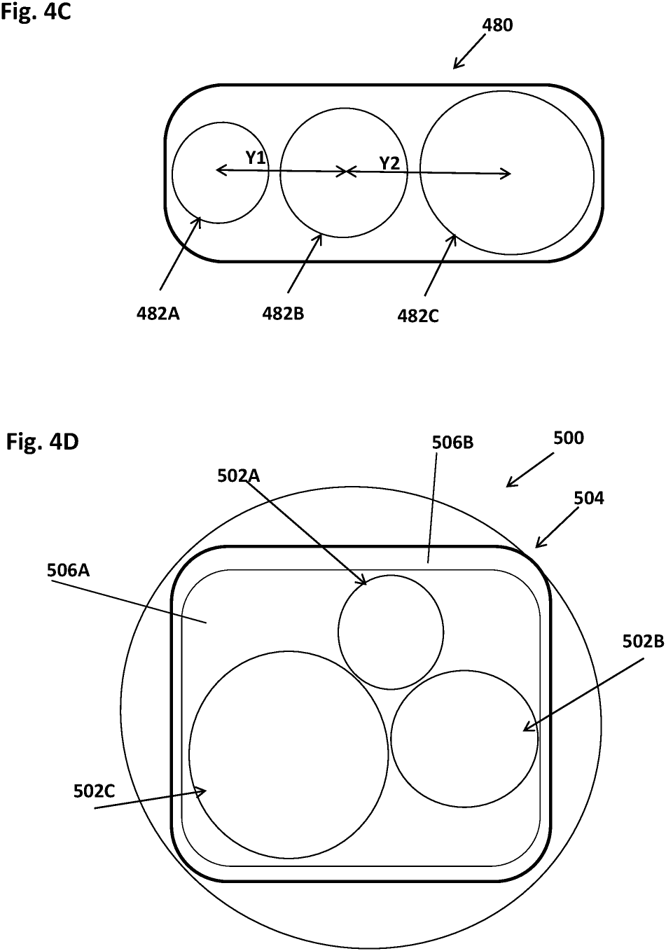

Reference is now made to FIG. 4A, which depicts a perspective view of a front and side imaging units at a tip of an endoscope, according to some embodiments depicted in FIG. 3. As shown in FIG. 4A, endoscope tip 400, includes a front imaging unit which faces a front view of endoscope tip 400, 410 and a side imaging unit 420 which faces a side view of endoscope tip 400. FIG. 4A shows endoscope tip 400 includes front and side imaging units, such as front imaging unit 120a, first side imaging unit 120b and second side imaging unit 120c which operation is disclosed at FIG. 3. Front imaging unit 410 includes three front optical lens assemblies, 412A, 412B and 412C. Each of the front optical lens assemblies 412A-C may be associated with a separate front image sensor, or with a common front image sensor (not shown). Front optical lens assemblies 412A-C are different from each other with respect of size, shape, composition and/or position. Each of the front optical lens assemblies 412A-C has a different focal length and depth of field, whereby each of the front lens assemblies is configured to thereby provide a sharp and focused image (still images and/or video stream images) at a different range of distances. The images obtained from the front lens assemblies may than be further processed (for example, by interpolation or superposition) to provide an enhanced, clear and focused image of an object and/or region of interest covered by the front imaging unit 410. In some embodiments, the provided image from the front imaging unit is a multifocal, 3D image, which allows a user to perceive a clear and focused image at varying depth of fields and/or working distances. For example, in some exemplary embodiments, a first front optical lens assembly may have a depth of view in the range of about 2-9 millimeters, a second front optical lens assembly may have a depth of field (view) distance in the range of about 5-20 millimeters and a third front optical lens assembly may have a depth of view in the range of about 15-500 millimeters. Thus, by obtaining simultaneous images from each of these front optical lens assemblies, a clear, multi-focal image may be constructed, which can provide a clear and focused image in a wide range of depth of view, such as, in the range of 2-500 millimeters or 2-200 millimeters. Further, in addition to obtaining a sharp and focused image over a wide range of depth of fields, a stereoscopic or 3D image may be obtained. In some embodiments, as shown in FIG. 4A, the arrangement of the front lens assemblies 412A-C of front imaging unit 410 may be in the form of a triangle, such that the distance between the center of the front optical lens assemblies may be minimal, to minimize parallax effect, as further detailed below. In some embodiments, the direction of view of front optical lens assemblies 412A-C may be similar. In some embodiments, the field of view of the front optical lens assemblies may be similar.

As further shown in FIG. 4A, side imaging unit 420 includes three side optical lens assemblies, 422A, 422B and 422C. Each of the side optical lens assemblies 422A-C may be associated with a separate side image sensor, or with a common side image sensor (not shown). Side optical lens assemblies 422A-C are different from each other with respect of size, shape, composition and/or position. Each of the side optical lens assemblies 422A-C has a different focal length and depth of field, whereby each of the side optical lens assemblies 422A-C is configured to thereby provide a sharp and focused image (still images and/or video stream images) at a different range of distances. The images obtained from the side optical lens assemblies 422A-C may than be further processed (for example, by interpolation or superposition) to provide an enhanced, clear and focused image of the object and or region of interest covered by the side imaging unit 420. In some embodiments, the provided image from the side imaging unit 420 is a multifocal, optionally, 3D image, which allows a user to perceive a clear and focused image at varying depth of fields. For example, in some exemplary embodiments, a first side optical lens assembly may have a depth of view in the range of about 2-9 millimeters, a second side optical lens assembly may have a depth of field (view) distance in the range of about 5-20 millimeters and a third side optical lens assembly may have a depth of view in the range of about 15-200 millimeters. Thus, by obtaining simultaneous images from each of these side optical lens assemblies, a clear, multi-focal image may be constructed, which can provide a clear and focused image in a wide range of depth of fields, such as, in the range of 2-200 millimeters. Further, in addition to obtaining a sharp and focused image over a wide range of depth of views, a stereoscopic or 3D image may optionally be obtained. In some embodiments, as shown in FIG. 4A, the arrangement of the side lens assemblies 422A-C of side imaging unit 420 may be linear, such as vertical or horizontal arrangement, relatively to a longitudinal axis of endoscope tip 400 (as shown in FIG. 3., longitudinal axis A). In some embodiments, the direction of view of side optical lens assemblies 422A-C may be similar. In some embodiments, the field of view of the side optical lens assemblies 422A-C may be similar. In some embodiments, the images obtained from the front optical lens assemblies 412A-C and side optical lens assemblies 422A-C may than be further processed (for example, by interpolation or superposition) to provide an enhanced, clear and focused image of the entire region of interest covered by both front and side imaging unit 410, 420, respectively.

Reference is now made to FIG. 4B, which shows a zoom-in front view of a front imaging unit, according to FIG. 4A. The front imaging unit 450 illustrated in FIG. 4B includes three front optical lens assemblies 452A-C. Each of the front optical lens assemblies may be associated with a respective front optical sensor or a common front sensor maybe associated with two or more of the front optical lens assemblies. As shown in the example presented in FIG. 4B, the front optical lens assemblies are 452A-C arranged in a triangle shape, whereby distances (X1, X2 and X3) between the centers of the front optical lens assemblies are minimal, so as to minimize the parallax effect. In some embodiments, each of the distances X may be determined according to a minimal depth of field between front imaging unit 450 to an object/region of interest (front imaging unit 450, 120a is positioned on a front surface 146 of the endoscope shaft distal section 400, 112 as shown in FIG. 3). In some embodiments, the minimal distance (i.e., X1 and/or X2 and/or X3) between the front optical lens assemblies 452 is smaller than the minimal distance between the smallest front optical lens assembly (i.e., the lens with the shortest depth of field) and the region of interest. In some exemplary embodiments, the distance between the front optical lens assemblies’ centers is less than about 2-3 millimeter.

In some embodiments, as illustrated in FIG. 4B, the front optical lens assemblies 452A-C are different at least with respect of size and hence with respect of their depth of field (and/or working distance). For example, front optical lens assembly 452A, has the smallest diameter (for example, front optical lens assembly 452A may be associated with a sensor having a diameter in a range of 0.5-2.5 millimeters) and the shorter (closest) depth of field distance (for example, a range of 2-9 millimeters), front optical lens assembly 452B has a medium diameter (for example, front optical lens assembly 452B may be associated with a sensor having a diameter in a range of 2-3.5 millimeters) and a medium depth of field distance (for example, in the range of about 5-20 millimeters) and front optical lens assembly 452C has the largest diameter (for example, front optical lens assembly 452C may associate with a sensor having a diameter in a range of 3-4.5 millimeters) and the longer depth of field distance (for example, in the range of about 15-500 millimeters). Thus, the imaging unit illustrated in FIG. 4B can provide a sharp and clear image (still or video) at a varying depth of field (for example, in a wide range of 2-200 millimeters), wherein the image is a stereoscopic image or 3D image and is obtained by processing, in real-time the images obtained from each of the lens assemblies. By such an advantageous setting, a clear, non-distorted image (optionally 3D image), showing details at varying depth of fields (such as in the range of about 2-500 millimeters) is obtained, while maintaining a small form factor, capable of being located within the limited space of the endoscope tip. In some embodiments, the front optical lens assemblies have a similar or identical direction of view (i.e., they all point to the same direction). In some embodiments, the front optical lens assemblies may reside in the same plan.

Reference is now made to FIG. 4C, which shows a zoom-in view of an imaging unit, according to FIG. 4A. Imaging unit 480 may be a front imaging unit, such as front imaging unit 410 or a side imaging unit, such as side imaging unit 420. Imaging unit 480 includes three optical lens assemblies, 482A, 482B and 482C. Each of the optical lens assemblies 482A-C may be associated with a separate image sensor, or with a common image sensor (not shown). As shown in the Example presented in FIG. 4C, the optical lens assemblies 482A-C are arranged in vertical or horizontal form. In some embodiments, distances Y1 and Y2 between centers of the optical lens assemblies 482A to 482B, and 482B to 482C, respectively, may be determined according to a minimal depth of field of imaging unit 480. In some embodiments, the minimal distance Y1 and/or Y2 between the optical lens assemblies 482 is smaller than the minimal distance between the smallest optical lens assembly (i.e., the lens with the shortest depth of field), and a region of interest. In some embodiments, as illustrated in FIG. 4C, the optical lens assemblies are different at least with respect of size and hence with respect of their depth of field. For example, optical lens assembly 482A, has the smallest diameter (for example, optical lens assembly 482A may associate with a sensor having a diameter in a range of 0.5-2.5 millimeters) and the shorter (closest) depth of field distance (for example, a range of 2-9 millimeters), optical lens 482B has a medium diameter (for example, optical lens assembly 482B may associate with a sensor having a diameter in a range of 2-3.5 millimeters ) and a medium depth of field distance (for example, in the range of about 5-20 millimeters), and lens 482C has the largest diameter (for example, optical lens assembly 482C may associate with a sensor having a diameter in a range of 3-4.5 millimeters) and the longer depth of field distance (for example, in the range of about 15-500 millimeters). Thus, imaging unit 480 can provide a sharp and clear image (still or video) at a varying depth of fields (for example, in a wide range of 2-500 millimeters or 2-200 millimeters), wherein the image is a stereoscopic image or 3D image and is obtained by processing, in real-time, based on the images obtained from each of the lens assemblies 482A-C. By such an advantageous setting, a clear, non-distorted image, optionally a 3D image, showing details at varying depth of views is obtained, while maintaining a small form factor, capable of being located within the limited space of the endoscope tip. In some embodiments, the optical lens assemblies have a similar or identical direction of view (i.e., they all point to the same direction). In some embodiments, the lens assemblies may reside in the same plan. In some embodiments, the internal arrangement of the three optical lens assemblies 482A-C may vary, such that optical lens assembly 482A may be placed between optical lens assemblies 482B and 482C, optical lens assembly 482C may be placed between optical lens assemblies 482B and 482A, and the like.

Reference is now made to FIG. 4D, which shows a zoom-in view of an imaging unit, according to some FIG. 4A. The imaging unit 500 illustrated in FIG. 4D includes three optical lens assemblies 502A-C. The optical lens assemblies 502A-C are associated with one optical sensor 504, having an active area region 506A and mechanical area portion 506B. As shown in the Example presented in FIG. 4D, the optical lens assemblies 502A-C are arranged in a triangle shape, whereby the distances between the centers of the optical lens assemblies 502A-C is minimal, so as to minimize the parallax effect. Obtaining a minimal distance between the centers of lens assemblies may achieved, inter alia, due to the use of a common optical sensor, whereby the optical lenses assemblies can be placed in close proximity on the surface of the optical sensor, wherein each lens assembly can utilize various portions of the active area region of the sensor. In some embodiments, as illustrated in FIG. 4D, the optical lens assemblies are different at least with respect of size and hence with respect of their depth of field (and/or working distance). For example, optical lens assembly 502A, has the smallest diameter (for example, optical lens assembly 502A may associate with a sensor having a diameter in a range of 0.5-2.5 millimeters) and the shorter (closest) depth of field distance (for example, a range of 2-9 millimeters), optical lens assembly 502B has a medium diameter (for example, optical lens assembly 502B may associate with a sensor having a diameter in a range of 2-3.5 millimeters) and a medium depth of field distance (for example, in the range of about 5-20 millimeters) and optical lens assembly 502C has the largest diameter (for example, optical lens assembly 502C may associate with a sensor having a diameter in a range of 3-4.5 millimeters) and the longer depth of field distance (for example, in the range of about 15-500 millimeters). Thus, imaging unit 500 can provide a sharp and clear image (still or video) at a varying depth of fields (for example, in a wide range of 2-200 millimeters), wherein the image is a stereoscopic image or 3D image and is obtained by processing, in real-time the images obtained from each of the optical lens assemblies 502A-C. By such an advantageous setting, a clear, non-distorted 3D image, showing details at varying depth of fields is obtained, while maintaining a small form factor, capable of being located within the limited space of the endoscope distal tip. In some embodiments, the optical lens assemblies 502A-C have a similar or identical direction of view (i.e., they all point to the same direction). In some embodiments, the optical lens assemblies 502A-C may reside in the same plan. In some embodiments, the optical lens assemblies 502A-C may be placed at a relative angle relative to each other, to make maximal use of the active area region of sensor 504, while maintaining minimal distance between the optical lens assemblies.

According to some embodiments, optical lens assemblies of an imaging unit may be different from each other with respect of one or more of their properties, including, but not limited to: size, composition, type, working distance, focal length, depth of field, position, location, plane, distance, and/or topology. Each possibility is a separate embodiment.

According to some embodiments, different imaging units may be identical, similar or different with respect to the optical lens assemblies thereof. For example, the imaging units may have different or similar lens assemblies types, different or similar lens assemblies number, different or similar lens assemblies configuration, different or similar lens assemblies topology, and the like.

In some embodiments, one or more of the optical lens assemblies may have autofocus capabilities.

In some embodiments, the field of view (FOV) of the optical lens assemblies of an imaging unit may be similar.

In some embodiments, in order to provide a short depth of view (for example, a distance of 2-10 millimeters), the optical lens assembly and associated sensor may be in the size range of less than about 2.5 millimeters.

In some embodiments, in order to provide a medium depth of view (for example, distance of 5-20 millimeters), the optical lens assembly and associated sensor may be in the size range of less than about 3.5 millimeters.

In some embodiments, in order to provide a longer depth of view (for example, distance of 15-200 millimeters), the lens assembly and associated sensor may be in the size range of less than about 4.5 millimeters.

In some embodiments, each of the imaging units may include one or more illumination component. In some embodiments, each optical lens assembly may include a discrete illumination component.

According to some embodiments, there is provided an endoscope having at a distal end/tip thereof three imaging units: a front facing imaging unit, a first side-facing imaging unit and a second side facing imaging unit, wherein the front facing imaging unit includes three optical lens assemblies associated with at least one optical sensor and each of the side facing imaging units includes at least two optical lens assemblies associated with at least one optical sensor.

According to some embodiments, the endoscope distal tip disclosed herein, i.e., a tip having a plurality of imaging units, at least one of the imaging units having at least two optical lens assemblies associated with at least one optical sensor may be used in various types of endoscopes, such as, flexible, semi-rigid and rigid endoscopes.

According to some exemplary embodiments, flexible endoscope may include such endoscopes for use in renal procedures, urological procedures, nasal procedure, orthopedic procedures, and the like. In some embodiments, such endoscopes may include imaging units having two optical lens assemblies, providing a combined depth of field images (i.e. image obtained from the two optical lens assemblies, each having a different depth of field capabilities) in the range of, for example, 2-50 millimeters.

According to some embodiments, endoscopes for use in procedures such as, for example, colonoscopy, genecology, laparoscopy, may include imaging units having three optical lens assemblies, providing a combined depth of field images (i.e. image obtained from the three optical lens assemblies, each having a different depth of field capabilities) in the range of, for example, 2-200 millimeters.

In some embodiments, the endoscope distal tip may include any combination of imaging units. For example, in some embodiments, the endoscope distal tip may include a front facing imaging unit having three optical lens assemblies, associated with at least one optical sensor and two side facing imaging units, each having three optical lens assemblies, associated with at least one optical sensor. For example, in some embodiments, the endoscope distal tip may include a front facing imaging unit having three optical lens assemblies, associated with at least one optical sensor and two side facing imaging units, each having two optical lens assemblies, associated with at least one optical sensor. For example, such a setting may be useful for obtaining front, sharp and clear images at a wide depth of field and side images obtained from two optical lens assemblies, which are useful for stitching or other secondary medical procedures during the main endoscopic procedure.

According to some embodiments, there is provided a method for obtaining a focused image of a body cavity at a varying depth of field relative to an endoscope tip, the method includes inserting into the body cavity an endoscope shaft having tip at the distal section thereof, wherein the distal tip includes plurality of imaging units, at least one of said imaging units includes at least two optical lens assemblies and at least one optical sensor associated with the at least two optical lens assemblies, wherein each optical lens assembly comprises a different depth of field; and obtaining or generating a focused image of the body cavity at a varying depth of field relative to the distal tip.

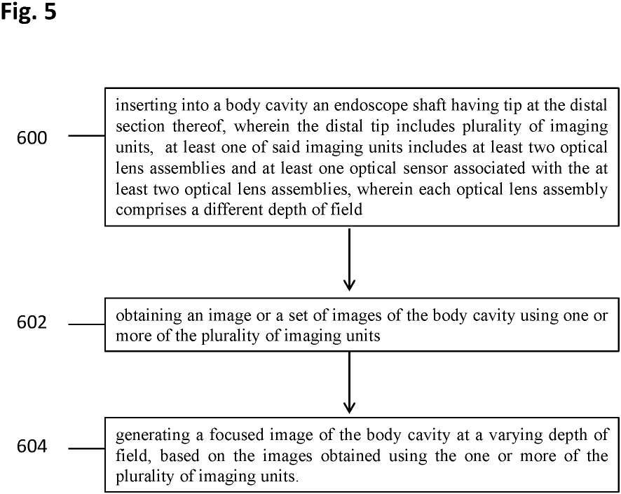

Reference is now made to FIG. 5, which illustrates steps in a method for obtaining a focused image of a body cavity/body region/area of interest at varying depth of field, according to some embodiments. As shown in FIG. 5, at step 600, an endoscope shaft having tip at the distal section thereof, wherein the distal tip includes plurality of imaging units, at least one of said imaging units includes at least two optical lens assemblies and at least one optical sensor associated with the at least two optical lens assemblies, wherein each optical lens assembly comprises a different depth of field is inserted into the body cavity. At step 602, one or more images (or video) are obtained using at least one of the imaging units. At step 604, a focused image of the body cavity at a varying depth of field, is generated/produced (for example, on a processing unit), based on the images obtained using the one or more of the plurality of imaging units in step 602 and the generated focused image may be displayed.

In some embodiments, the varying depth of field may be in any desired range, based on the type of endoscope and the medical procedure. In some embodiments, the number, type, size, topology and/or composition of the various optical lens assemblies of the various imaging units can determined the combined, varying wide range of depth of field over which a focused image is obtained. In some embodiments, varying depth of field may be in the range of about 1-750 millimeters. In some embodiments, varying depth of field may be in the range of about 2-500 millimeters. In some embodiments, varying depth of field may be in the range of about 2-300 millimeters. In some embodiments, varying depth of field may be in the range of about 2-200 millimeters. In some embodiments, varying depth of field may be in the range of about 1-200 millimeters. In some embodiments, varying depth of field may be in the range of about 2-100 millimeters. In some embodiments, varying depth of field may be in the range of about 1-50 millimeters.

In some embodiments, the generated focused image may be generated in real time, by a processing unit (for example, processing unit of a main control unit), which is configured to generate the focused image based on the images obtained from the at least two optical lens assemblies of the imaging units. In some embodiments, the processing unit is configured to generate the focused image by interpolation and/or superposition of the images obtained from the different optical lens assemblies.

According to some embodiments, the focused image generated by the method is a multi-focal image. In some embodiments, the generated focused image is a 3D image.

According to some embodiments, the method may further include presenting or displaying the focused image and/or the individual images obtained from one or more optical lens assemblies.

According to some embodiments, there is provided a method of using an endoscope having a tip as disclosed herein, for obtaining an image (still or video) of a body cavity at a varying depth of field.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system’s registers and/or memories, into other data similarly represented as physical quantities within the computing system’s memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus. In some embodiments, a computer may include of the apparatuses may include FPGA, microcontrollers, DSP and video ICS.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

As used herein, according to some embodiments, the terms “substantially” and “about” may be interchangeable.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1-35. (canceled)

36. An endoscope distal tip comprising:
a plurality of imaging units, at least one of said imaging units comprises at least two optical lens assemblies and at least one optical sensor associated with the at least two optical lens assemblies, wherein each optical lens assembly comprises a different depth of field, thereby allowing to obtain a focused image of a body part at a varying depth of field relative to the tip.

37. The endoscope tip according to claim 36, wherein said at least one optical sensor is associated with an image processor; wherein the sensor is selected from CMOS and CCD.

38. The endoscope tip according to claim 36, wherein the distal tip further comprises one or more illumination components associated with said at least one imaging unit.

39. The endoscope tip according to claim 36, wherein each optical assembly of the at least two optical lens assemblies is associated with a respective optical sensor.

40. The endoscope tip according to claim 36, wherein at least one of the imaging units comprises at least three optical lens assemblies.

41. The endoscope tip according to claim 36, comprising at least two imaging units, comprising a front facing imaging unit and a first side facing imaging unit.

42. The endoscope tip according to claim 41, wherein the at least two imaging units further comprise a second side-imaging unit, wherein the first imaging unit and the second imaging unit are positioned on opposite sides of the endoscope distal tip.

43. The endoscope tip according to claim 41, wherein the at least two imaging units provide at least about 270 degrees horizontal field of view (FOV) of a region of interest within an anatomical body cavity into which the distal tip is inserted, wherein at least one of the field of views comprises a multi focal image.

44. The endoscope tip according to claim 41, wherein each of the at least two of the imaging units comprises at least three optical lens assemblies.

45. The endoscope tip according to claim 40, wherein the at least three optical lens assemblies of an imaging unit are arranged in the form of a triangle, wherein the distance between the center of the least three optical lenses is smaller than about 3 millimeters.

46. The endoscope tip according to claim 45, wherein a minimal distance between the at least three optical lens assemblies of an imaging unit is smaller than a minimal distance between an optical lens assembly with a shortest depth of field and the region of interest.

47. The endoscope tip according to claim 40 wherein the at least three optical lens assemblies of an imaging unit are arranged in horizontal or vertical line relative to each other.

48. The endoscope tip according to claim 40, wherein a first optical lens assembly is configured to provide a focused image in a depth of field of about 2-10 millimeters.

49. The endoscope tip according to claim 48, wherein the minimal distance between the optical lens assemblies of an imaging unit is smaller than the minimal distance between the first optical lens assembly having the shortest depth of field and the region of interest.

50. The endoscope tip according to claim 40, wherein a second optical lens assembly is configured to provide a focused image in a depth of field of about 5-20 millimeters and/or wherein a third optical lens assembly is configured to provide a focused image in a depth of field of about 15-500 millimeters.

51. The endoscope tip according to claim 36, wherein the varying depth of field relative to the tip is in the range of about 2-500 millimeters.

52. An endoscope comprising the tip according to claim 36 at a distal section of an elongated shaft of the endoscope.

53. A medical imaging system comprising the endoscope of claim 52, and a display configured to display the images and/or video generated by the one or more of the imaging units.

54. The medical imaging system according to claim 53, further comprising a processing unit configured to receive images obtained from the at least two optical lens assemblies of the imaging units and generate in real time a focused image at varying depth of fields.

55. The medical imaging system according to claim 53, wherein the generated focused image is a 3D image of a body cavity, in which the endoscope tip resides.

56. A method for obtaining a focused image of a region of interest at a varying depth of fields relative to an endoscope distal tip, the method comprising:
inserting into the region of interest an endoscope shaft comprising the endoscope distal tip, said endoscope distal tip comprising a plurality of imaging units, at least one of said imaging units comprises at least two optical lens assemblies and at least one optical sensor associated with the at least two optical lens assemblies,
wherein each optical lens assembly comprises a different depth of field; and generating a focused image of the region of interest at a varying depth of field relative to the distal tip.

57. The method according to claim 56, wherein the varying depth of field is in the range of about 2-500 millimeters; and/or
wherein the generated focused image is generated in real time by a processing unit configured to generate said focused image based on the images obtained from the at least two optical lens assemblies of the imaging units.
